(19) United States
(12) Patent Application Publication
(10) Pub. No.: US 2025/0345624 A1
(43) Pub. Date: Nov. 13, 2025

(54) LED-Enhanced Vibrating Micro-Needling Device

(71) Applicant: Olaide Soetan, Springfield Gardens, NY (US)

(72) Inventor: Olaide Soetan, Springfield Gardens, NY (US)

(21) Appl. No.: 19/187,950

(22) Filed: Apr. 23, 2025

Related U.S. Application Data
(60) Provisional application No. 63/637,717, filed on Apr. 23, 2024.

(51) Int. Cl. A61N 5/06

(52) U.S. Cl. CPC A61N 5/0617; A61N 2005/0626; A61N 2005/0632; A61N 2005/0644; A61N 2005/0652; A61N 2005/0663

(57) ABSTRACT

A handheld treatment device that combines microneedling, vibration, and red-light therapies to enhance skin rejuvenation and hair growth. The device includes a housing with a handle portion and a needle bed portion, the latter configured to contain a microneedle cartridge and a red-light therapy module. The handle portion contains a control module, one or more user inputs, and a vibration module. The combination of microneedling, vibration, and red-light therapies allows a user to simultaneously target structural and biochemical skin and hair issues with a single handheld device.

### TECHNICAL FIELD OF THE DISCLOSURE

The invention of the present disclosure is related generally to the field of skincare devices. More specifically, the present disclosure relates to a relates to a skincare and haircare device providing simultaneous microneedling, red-light therapy, and vibration massage therapy for enhanced collagen induction, improved circulation, and tissue regeneration.

### INTRODUCTION

Microneedling is a widely used technique in dermatology designed to promote collagen production, improve skin texture, and enhance transdermal absorption of skincare products. By puncturing the epidermal (and sometimes dermal) layers of the skin, microneedling causes controlled micro-injuries that spur natural healing processes, which improve the appearance and health of skin.

Red-light exposure therapy (also known as photobiomodulation) is another popular skincare treatment that promotes collagen synthesis, stimulates cellular activity, and reduces inflammation. Red light has a wavelength typically between 620-750 nm, and is known improve skin tone and texture, minimize the appearance of pores and scars, and accelerate wound healing.

Vibration massage therapy is a third treatment that has been shown to encourage regenerative processes by promoting muscle relaxation and microcirculation in tissues, including the skin. Combining vibration massage therapy with microneedling allows the effects of both vibration massage and microneedling therapies to extend deeper into the skin, imparting further benefits and encouraging faster, more evident results.

Despite their individual therapeutic benefits, microneedling, vibration treatment, and red-light therapy are rarely carried out with a single device. Enhancement of microneedling treatments with vibration and red-light therapy will accelerate healing and hair growth processes, further amplify collagen production and fibroblast proliferation, boost ATP production, and increase circulation, all resulting in firmer, more elastic, healthier skin. What's more, red light has been shown to reduce inflammation, which is a common side effect of microneedling treatments.

Currently, there are no devices designed for simultaneous microneedling, vibration therapy, and red-light therapy. Combination of these non-invasive treatments in a single device will maximize the benefits and minimize the side effects of each treatment. The use of three simultaneous treatments will serve to promote collagen production through mechanical, biochemical, and physical stimulation, improve skin texture, stimulate hair follicle activity, accelerate healing, and reduce inflammation and side effects.

Therefore, there exists a need for a skincare device that targets structural and biochemical skin issues, while enhancing blood flow and lymphatic drainage. Simultaneous execution of red-light therapy, vibration therapy, and microneedling provides a comprehensive rejuvenation treatment, all in a controlled and efficient single device.

### SUMMARY

The present invention provides a handheld device that integrates microneedling, vibration, and red-light therapy. This multifunctional device enhances therapeutic outcomes by promoting regenerative processes, including collagen production and hair growth, improving skin texture, accelerating healing, with minimized side effects from treatment.

The device comprises a handle, a microneedle cartridge comprising a plurality of microneedles, a vibration module, a red-light therapy module, a control module, a power module, and an outer housing. The red-light therapy module comprises at least one light emitting diode configured to emit red light, and is positioned adjacent to the microneedle cartridge. The vibration module causes oscillatory motion of the microneedle cartridge along a longitudinal axis. The control module comprises a control interface for activating the vibration module and the red-light therapy module, either simultaneously or separate from one another. The microneedle cartridge may be irremovably coupled to the other components of the device, or may be detachable and replaceable. The microneedle cartridge allows the red light to pass through it. The device is battery-powered and rechargeable, and emits red light with a wavelength between 600 nm and 750 nm. The control module includes pre-set treatment modes combining microneedling, vibration, and red-light therapy, and may also comprise a wireless communication module for connectivity with an external device. The device may also include an end cap, which may be configured to cover the microneedle cartridge.

Also disclosed herein is a method of treating skin. The method comprises the steps of applying a microneedling treatment to a skin surface using a device while simultaneously applying vibration therapy and red-light therapy to the same skin surface using a red-light module integrated into the device.

### BRIEF DESCRIPTION OF THE DRAWINGS

FIG. 1 is a perspective view showing a fully assembled embodiment of the device.

FIG. 2 is a cross-section showing the internal components of the device.

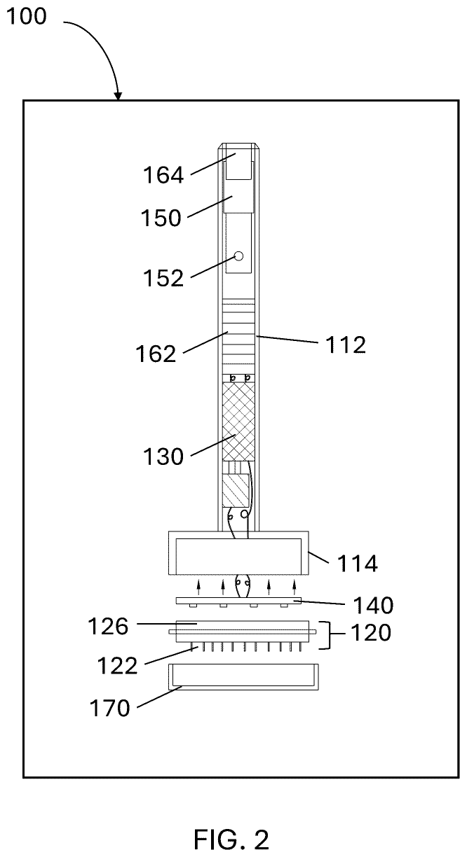

FIG. 3 is an exploded perspective view showing a partially disassembled embodiment of the device.

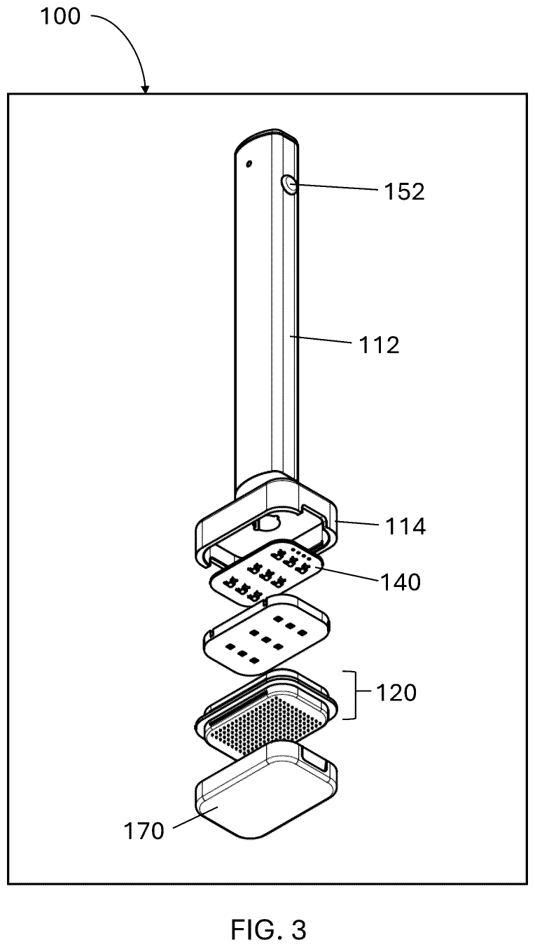

FIG. 4 is an exploded perspective view of an embodiment of the device, showing its internal electronic components.

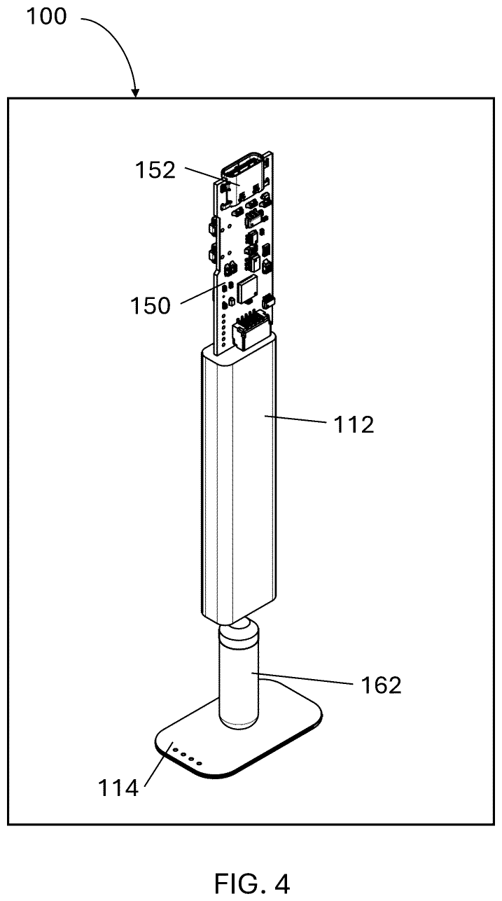

FIG. 5 is a perspective view of an exemplary microneedle cartridge, with a close-up of the array.

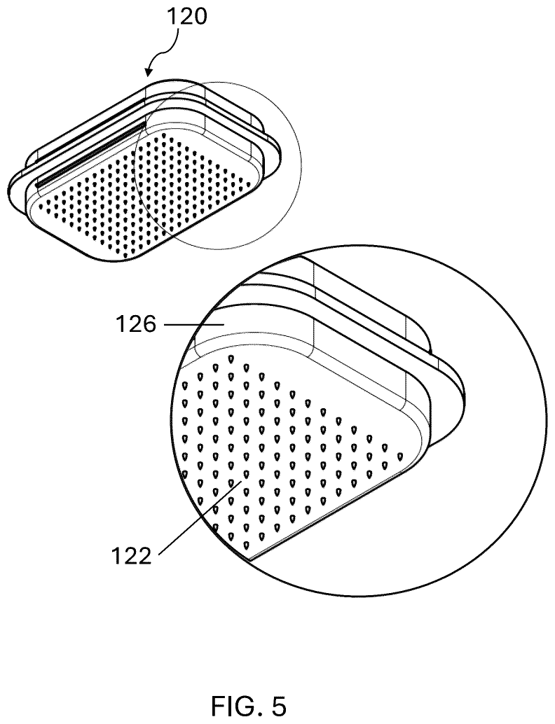

### DETAILED DESCRIPTION

Throughout the specification, wherever practicable, like structures will be identified by like reference numbers. In some figures, components such as electrical connections or fasteners have been omitted for clarity in the drawings. Although the present device and method have been described in terms of various embodiments, it is to be understood that such disclosure is not intended to be limiting. The embodiments are intended to be illustrative, not limiting. Various alterations and modifications will be readily apparent to those of skill in the art. Accordingly, it is intended that the claims be interpreted to cover all alterations and modifications that fall within the spirit and scope of the invention.

Aspects of the present disclosure may be used to treat a user's skin and/or hair using a handheld device. The present disclosure may be used to simultaneously provide the benefits of microneedling, vibration, and red-light therapies. The device of the present disclosure may be used for both skincare and hair restoration treatments. The examples discussed herein largely focus on dermal use, however use of the device for hair rejuvenation, growth, and regrowth purposes is also within the scope and spirit of this disclosure.

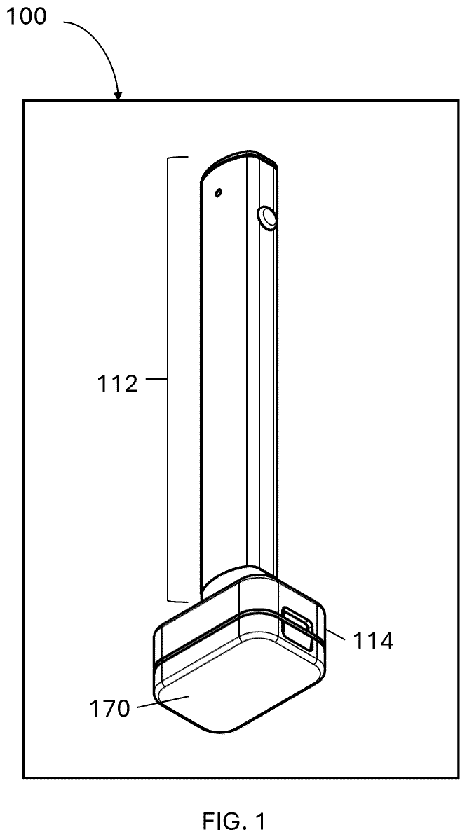

Turning to FIG. 1, the device of the present disclosure may include a skincare device 100 with the following components: a housing 110, a microneedle cartridge 120, a vibration module 130, a red-light therapy module 140, a control module 150, a power module 160, and a removable cover 170. In an embodiment, the housing 110 may be designed, sized, or otherwise configured to allow for comfortable handheld use of the device 100. The housing 110 may be a single continuous piece, or may be formed from multiple housing components configured to interface with one another. In an embodiment, the housing 110 may comprise a handle housing 112 and a needle bed housing 114. The handle housing 112 may be ergonomically configured to enable handheld control of the device 100, and may also be configured to contain various components, including but not limited to the vibration module 130, the control module 150, and the power module 160. The handle housing 112 may have any suitable shape known to a person of ordinary skill in the art. In a preferred embodiment, the handle housing 112 is rod shaped. In an alternative embodiment, the handle housing 112 has a rectangular prism shape.

The needle bed housing 114 may be configured to house one or more treatment modules, including at least the microneedle cartridge 120 and the red-light therapy module 140. In an embodiment, the needle bed housing 114 may contain a retention mechanism 116 for securely engaging one or more components including at least the microneedle cartridge 120 and the red-light therapy module 140. The needle bed housing 114 may have any suitable shape known to a person of ordinary skill in the art, and may further have a skin contact surface area 118 and a needle bed housing height 119. In an embodiment, the skin contact surface area 118 may be a flat surface defined by any two-dimensional shape. Preferably, the skin contact surface area 118 has a width that is larger than the width of the handle housing 112. In an embodiment, the needle bed housing 114 may have a cylindrical shape. In such an embodiment, the skin contact surface area 118 is a circular shape. In an alternative embodiment, the skin contact surface area 118 may have a square or rectangular shape. In yet another alternative embodiment, the skin contact end may be configured to allow variably targeted use of the device 100 depending on the angle between the skin contact surface area 118 and the skin of a user, for example with a tapered end and a broader end.

The needle bed housing 114 may be rigidly coupled to the handle housing 112 such that when a vibration module 130 is causing one or more components of the device 100 to move relative to one another, the needle bed housing 114 and the handle housing 112 remain in the same relative position. In an alternative embodiment, the needle bed housing 114 and the handle housing 112 may be coupled to one another in a manner that allows the a user to adjust the position of the needle bed housing 114 relative to the handle housing 112 along an longitudinal axis running substantially parallel to a length of the device 100.

The device 100 may comprise a microneedle cartridge 120. The microneedle cartridge 120 may be detachable and replaceable, and may comprise a plurality of needles arranged in an array 122. The needles may be arranged to penetrate a user's skin to a predetermined depth. The needles may be formed from any suitable material known in the art, most preferably stainless steel, titanium, or polymer-coated alloys. The needles may be solid or hollow, cylindrical or conical in shape, and may have any suitable length depending on a user's desired depth of penetration. It is anticipated that the most common needle lengths will be less than approximately 2.5 mm, however needle lengths outside this range are within the scope of this disclosure. In some embodiments, the microneedle cartridge 120 may include a depth-adjustment dial or other length adjustment mechanism that allows a user to set a needle penetration depth. The needle penetration depth may be electronically or manually adjusted through the control module 150, by changing the position of the microneedle cartridge 120 relative to the housing 110, or by installing a microneedle cartridge 120 with the user's preferred needle length. The array 122 may be a grid with evenly spaced or unevenly spaced needles.

The array 122 may be mounted on a rigid or semi-rigid base plate 126 that is configured to interface with the red-light treatment module 140. Base plate 126 may be solid, and may be made from a transparent or opaque material. In an embodiment, base plate 126 may have one or more openings configured to allow a light emitted by the red-light treatment module 140 to pass through the microneedle cartridge 120 and irradiate the treatment site simultaneously with microneedling.

The microneedle cartridge 120 may be reusable, or may be configured for single use. In an embodiment with a reusable microneedle cartridge, the device 100 may incorporate reinforced mechanical components and may be configured to be water-resistant or waterproof. In this embodiment, the microneedle cartridge 120 may be protected by a removable cover 170.

The device 100 may also contain a vibration module 130. The vibration module 130 may be disposed within the handle 112. In an embodiment, the vibration module 130 may contain a vibration motor 132 that causes the microneedle cartridge 120 to pulse the needles in and out of a treatment site at controlled intervals. In another embodiment, vibration module 130 may cause the device 100 to oscillate as a whole, without causing the microneedle cartridge 120 to move relative to the device 100. In a third embodiment, the vibration module 130 may cause the microneedle cartridge 120 to pulse and simultaneously cause the device 100 to vibrate.

When the vibration motor 132 causes the device 100 to oscillate, the device 100 moves in a sinusoidal path that varies according to user preferences. Most preferably, the device 100 vibrates at a frequency between 80 Hz and 250 Hz and an amplitude between 0.2 mm and 2.5 mm. These values are non-limiting examples, and frequencies and amplitudes outside of this range are within the scope and spirit of this disclosure. The frequency, amplitude, and intensity of the vibrations may be adjusted by a user in real time, or may change according to a predefined treatment pattern, for example a pulsed, wave, or rhythmic vibration pattern. In some embodiments, the vibration module 130 may include a resistance or pressure sensor or a feedback control system.

When the device 100 is used to deliver microneedling therapy to the skin, the user may observe activation of regenerative processes. For example, microneedling creates controlled micro-injuries that trigger the body's natural healing response, leading to increased production of collagen and elastin. When applied to the scalp, microneedling stimulates dermal papilla cells at the base of hair follicles and promotes the release of growth factors such as VEGF (vascular endothelial growth factor), IGF-1 (insulin-like growth factor 1), and Wnt proteins, all of which are crucial for hair follicle regeneration and hair growth.

When the vibration module 130 causes the device 100 to deliver vibration therapy to the skin, additional benefits may be realized. Localized vibration may increase microcirculation to both skin and scalp, improving blood flow, oxygenation, nutrient delivery, and lymphatic drainage, and may also stimulate fibroblasts, resulting in collagen production. Additionally, skin massage that incorporates vibration is a non-invasive method of reducing inflammation, which both improves the look and feel of skin and provides an optimal environment for hair growth. Vibration therapy also provides a gentle massage, reducing muscle tension and encouraging relaxation.

In an embodiment, the vibration motor 132 may be coupled to the handle 112 via a vibration-isolating bracket or other means for attachment that promotes efficient transfer of oscillation toward the microneedle cartridge 120 and minimizes feedback into a user's hand. These may include shock absorbing materials and/or other vibration-dampening components which may isolate the vibration motor 132 and minimize vibration feedback into the handle 112. Additionally, a fail-safe may deactivate the vibration motor 132 in the event of excessive resistance or obstruction. The vibration module 130 may be controlled by the control module 150.

The device 100 may also comprise a red-light therapy module 140. The red-light therapy module 140 may be configured to emit visible light. In a preferred embodiment, the red-light therapy module 140 is configured to emit light with a wavelength between 620 nm and 700 nm. Most preferably, the red-light therapy module consists of one or more light-emitting diodes (LEDs) configured to emit light with a wavelength between 630 nm and 680 nm. The emitted red light may penetrate a user's epidermis and dermis, and may encourage collagen production and reduce inflammation. When applied to a user's scalp, the emitted red light may prolong the anagen (growth) phase of the hair cycle, supporting sustained hair growth and promoting regrowth.

The LEDs may be arranged in a grid or a cluster pattern, or in any pattern suitable for red-light therapy known in the art. In an alternative embodiment, the red-light therapy module 140 may emit a directed beam of light to target a specific treatment area. Each LED may emit a predetermined amount of light, and the total light output of the device 100 may be customizable according to a user's preferences.

The red-light therapy module 140 may be positioned within the needle bed housing 114, and may be in direct contact or in parallel with the microneedle cartridge 120. In an embodiment, the red-light therapy module 140 may vibrate in sync with the microneedle cartridge 120. In an alternative embodiment, the red-light therapy module may be stationary. The red-light therapy module 140 may be controlled by the control module 150.

The control module 150 may be housed in the handle 112 and may consist of a control interface 152. The control interface 152 may enable a user to access one or more settings of the device 100 via a user input. The control interface 152 may consist of physical buttons or levers, touch panels, or a digital display. In an alternative embodiment, the control interface 152 may be physically separate from the device 100, and the device 100 may contain a transmitter configured to send and receive information from the control interface 152.

The control module 150 may include a printed circuit board and electrical components forming a circuit designed to regulate operation of the device 100. These may include controllers, sensors, switches, status indicators, timers, noise-emitters, light-emitters, and electrical components known in the art. The control module 150 may be configured to cause the vibration module 130 to move the microneedle cartridge 120, to make the device 100 vibrate, or both, and may also be configured to activate the red-light therapy module 140. The control module 150 may be electrically coupled to any other component of the device 100.

The control module 150 may include one or more safety features, for example a temperature sensor configured to prevent overheating or burns, a timer to automatically deactivate the device after a period of no user input, a light diffuser to prevent hotspots, a blockage sensor that monitors the resistance encountered by the needles, a lockout mechanism to prevent inadvertent activation of the device 100, and/or any other safety component known in the art.

The power module 160 may include a power source 162. In an embodiment, the power source 162 is a rechargeable battery. The power module 160 may also include a charging interface 164, that enables a user to recharge the power source 162. The power module 160 may include safety components designed to prevent malfunction from overcharging, over-discharging, short-circuiting, or overheating of the device 100.

The device 100 may be configured to operate in a synchronous mode (microneedling, vibration, and red-light therapy) or asynchronously (microneedling, vibration, or red-light therapy, independently or in combination with one another). Optional features may include wireless connectivity for remote control or tracking treatment sessions via an app or other user interface.

Simultaneous application of microneedling, red-light therapy, and vibration massage therapy enhances the delivery of growth factors, cellular activation, and circulatory health-which can encourage hair restoration and skin rejuvenation processes.

What is claimed is:

1. A skincare device, comprising:
a handle;
a microneedle cartridge;
a vibration module;
a red-light therapy module comprising at least one light emitting diode configured to emit a red light;
a control module;
a power module; and
an outer housing.

2. The device of claim 1, wherein the vibration module causes oscillatory motion of the device.

3. The device of claim 1, wherein the red-light therapy module is positioned adjacent to the microneedle cartridge.

4. The device of claim 1, wherein the control module further comprises a control interface for independently or simultaneously activating the vibration module and the red-light therapy module.

5. The device of claim 1, wherein the microneedle cartridge is detachable and replaceable.

6. The device of claim 1, wherein the microneedle cartridge comprises a plurality of microneedles and a base plate.

7. The device of claim 1, wherein the microneedle cartridge allows the red light to pass through it.

8. The device of claim 1, wherein the device is battery-powered and rechargeable.

9. The device of claim 1, wherein the red light has a wavelength between 600 nm and 750 nm.

10. The device of claim 1, wherein the control module includes pre-set treatment modes combining microneedling, vibration, and red-light therapy.

11. The device of claim 1, wherein the control module allows a user to selectively and simultaneously activate one or more of a microneedle pulse, a vibration, and a red-light treatment.

12. The device of claim 1, further comprising a wireless communication module for connectivity with an external device.

13. The device of claim 1, further comprising an end cap, the end cap configured to cover the microneedle cartridge.

14. The device of claim 1, wherein the device is configured for use on the scalp to stimulate hair follicle activity and promote hair growth.

15. A method of treating skin, comprising the steps of:
simultaneously exposing a treatment area to a vibration therapy with a device; and
simultaneously exposing the treatment area to red-light therapy with the device.

16. The method of claim 15, further comprising applying a microneedling treatment to the treatment area with the device.

17. The method of claim 15, wherein the red-light therapy is generated by a red-light module within the device.

18. The method of claim 17, wherein the red-light module is configured to emit light at a wavelength between 600 nm and 750 nm.

19. The method of claim 15, wherein the treatment area is the scalp, and the method stimulates hair follicles and promotes hair regrowth.
